(19) United States
(12) Patent Application Publication
(10) Pub. No.: US 2021/0225194 A1
(43) Pub. Date: Jul. 22, 2021

(54) METHODS AND SYSTEMS FOR TREATING A SUBJECT USING NIRS FEEDBACK

(71) Applicant: Yale University, New Haven, CT (US)

(72) Inventors: Christopher Pittenger, Bethany, CT (US); Michelle Hampson, New Haven, CT (US)

(73) Assignee: Yale University, New Haven, CT (US)

(21) Appl. No.: 16/304,925

(22) PCT Filed: Jun. 8, 2017
(86) PCT No.: PCT/US2017/036532

§ 371 Date: Nov. 27, 2018

Related U.S. Application Data
(60) Provisional application No. 62/350,357, filed on Jun. 15, 2016.

(51) Int. Cl. G09B 19/00; A61B 5/00; A61B 5/16

(52) U.S. Cl. CPC G09B 19/00; A61B 5/0036; A61B 5/0075; A61B 5/6803; A61B 5/165; A61B 5/4064; A61B 5/743; A61B 5/4884

(57) ABSTRACT

One aspect of the invention provides a method of treating a subject diagnosed with one or more mental disorders selected from the group consisting of: anxiety disorders, mood disorders, trauma-associated disorders, psychotic disorders, and obsessive-compulsive disorder and related disorders. The method includes: performing near-infrared spectroscopy (NIRS) imaging of one or more regions of interest in the subject's brain; presenting a representation of the NIRS imaging to the subject; and while continuing to perform the performing and presenting steps: presenting a stimulus of anxiety or other symptomatology to the subject; instructing the subject to increase activity in the one or more regions of interest; and instructing the subject to decrease activity in the one or more regions of interest.

### CROSS-REFERENCE TO RELATED APPLICATION

This application claims priority to U.S. Provisional Patent Application Ser. No. 62/350,357, filed Jun. 15, 2016. The entire content of this application is hereby incorporated by reference herein.

### BACKGROUND OF THE INVENTION

Obsessive-compulsive disorder (OCD) is common, with a 1-year prevalence of 1.3% and a lifetime morbid risk of 2.7% in the U.S. It is characterized by recurrent, intrusive, and distressing thoughts and/or repetitive behaviors that result in significantly impaired occupational and social functioning. Effective pharmacological and psychotherapeutic therapies are available; however, even when optimally delivered, such interventions only help 60-70% of patients. Even patients who are classified as treatment responders typically continue to experience substantial symptoms and often experience a fluctuating, relapsing disease course and a substantially reduced quality of life.

### SUMMARY OF THE INVENTION

One aspect of the invention provides a method of treating a subject diagnosed with one or more mental disorders selected from the group consisting of: anxiety disorders, mood disorders, trauma-associated disorders, psychotic disorders, and obsessive-compulsive disorder and related disorders. The method includes: performing near-infrared spectroscopy (NIRS) imaging of one or more regions of interest in the subject's brain; presenting a representation of the NIRS imaging to the subject; and while continuing to perform the performing and presenting steps: presenting a stimulus of anxiety or other symptomatology to the subject; instructing the subject to increase activity in the one or more regions of interest; and instructing the subject to decrease activity in the one or more regions of interest.

This aspect of the invention can have a variety of embodiments. The one or more regions interest can include one or more selected from the group consisting of: an orbitofrontal cortex, a frontal pole, and a basal ganglia. The one or more regions interest can include the subject's frontal pole.

The representation of the NIRS imaging can be a graphical representation. The graphical representation can be a chart.

The method can further include downsampling data from the NIRS imaging to between about 0.2 Hz and about 6 Hz.

The method can further include repeating all steps within a single session. The method can further include repeating all steps in a new session. The method can further include calibrating the NIRS imaging to image the one or more regions of interest in the subject's brain images in a previous session to ensure that similar brain regions are monitored in successive sessions.

The obsessive-compulsive disorder and related disorders can include one or more selected from the group consisting of: obsessive-compulsive disorder, body dysmorphic disorder, hoarding disorder, trichotillomania, excoriation, and skin-picking disorder.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1, consisting of Panels (A) and (B), depicts specific regions of the orbitofrontal cortex (OFC) and frontal pole region that respond to contamination-related stimuli on an individual subject basis. The distribution of these sites, across all subjects, is shown on the brain images in Panel (A). Panel (B) depicts the display seen during neurofeedback. A series of neutral images (one of which is depicted in Panel (B)) or contamination-related images were shown.

FIG. 2, Panel (A) depicts changes in resting state brain functional connectivity in individuals who received neurofeedback. Panel (B) shows regions where pre-neurofeedback resting-state functional connectivity predict clinical response to the neurofeedback. Panel (C) shows regions where changes in resting state functional connectivity correlated with clinical response to the feedback. These are subtly different points.

FIG. 3 depicts improvement in OCD symptoms following one or two fMRI neurofeedback sessions. All 5 patients improved clinically and tolerated the procedure well (indeed, several asked to continue it).

FIG. 4 depicts fNIRS measurement of frontal cortex perfusion changes upon presentation of images provocative of specific OCD symptom subtypes. All images are within subject contrasts between provocative stimuli and neutral pictures, derived from 5 OCD and 5 control subjects, analyzed using NIRS-SPM software and corrected for statistical significance across all recorded channels. (NIRS-SPM is a MATLAB®-based software program available at http://www.fil.ion.ucl.ac.uk/spm/ and http://bispl.weebly.com/nirs-spm.html#/ and is described in J. C. Ye et al., “NIRS-SPM: Statistical parametric mapping for near-infrared spectroscopy,” 44(2) NeuroImage 428-47 (2009).)

FIG. 5 depicts a raw signal from a single optode pair in response to different types of stimuli in a subject with OCD. Stimulus type (N: neutral, A: emotionally arousing, but not specific to OCD symptoms) C: contamination, X: checking, S: symmetry) of sequential stimulus blocks is shown by the heights of the black bars at the bottom. Neutral and contamination-related stimuli are highlighted by red and blue bars, respectively. The increased response of this channel to contamination-related stimuli is apparent; this channel is thus a good candidate neurofeedback seed.

FIG. 6 depicts a method of a method of treating a subject according to an embodiment of the invention.

FIG. 7 depicts a graphic display according to an embodiment of the invention.

FIG. 8 a system for NIRS imaging, feedback, and instruction according to an embodiment of the invention.

FIG. 9 depicts a between-group contrast of brain activation in the OFC/frontal pole in patients with OCD upon symptom provocation. This image provides a comparison between OCD and controls and includes a statistical analysis of data from a group of patients and controls, rather than just illustrative data on individual subjects (as in FIG. 4). This unambiguously and empirically identifies the appropriate brain region/NIRS channels to which neurofeedback should be optimally targeted.

FIG. 10 depicts data from a single patient with OCD. NIRS measurements of OFC/frontal pole perfusion over the 30 seconds of individual neurofeedback trials are shown. Trials in which the subject was instructed to increase OFC activity are shown in the upper panel; the blue line shows the average of such trials on the first day of training and the orange line shows the average on the second day. The subject showed only a modest ability to increase OFC/frontal pole activity on the first day, but this was significantly improved on the second day. The lower panel shows the same thing, but for trials on which the subject was instructed to decrease OFC/frontal pole activity. Here the effect is even clearer: on the first day (blue) this subject showed almost no ability control OFC activity, but on the second day there was a very clear, learned ability. Improvements in both panels are statistically significant. These data are from only a single subject, but he is from the target population (OCD), and he very clearly learned from the biofeedback protocol.

FIG. 11 depicts initial data from a controlled fMRI neurofeedback study. OCD patients (n=6) showed symptom improvement (as measured using the Yale-Brown Obsessive Compulsive Scale (Y-BOCS)) after treatment according to an embodiment of the invention that continues to grow over the subsequent month, relative to patients who received no neurofeedback.

FIG. 12 depicts results from an n=1 study of a subject subclinical OCD symptoms, who underwent 2 days of NIRS neurofeedback. Using a procedure analogous to the fMRI neurofeedback work illustrated in FIG. 1, Applicant presented the subject with a series of contamination images at baseline, and then with a different but matched set of similar images a few days after neurofeedback. Reported anxiety/discomfort associated with these images was reduced after neurofeedback

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Functional neuroimaging studies suggest that regions of frontal cortex and various subcortical structures may play a role in the pathophysiology of OCD. Positron emission tomography (PET) studies have revealed abnormally high metabolic activity in patients in the orbitofrontal cortex (OFC), anterior cingulate cortex (ACC), and caudate nucleus. Single photon emission computed tomography (SPECT) studies have similarly indicated dysfunction in both the OFC and caudate nucleus. The medial OFC (mOFC) has been reported to be metabolically hyperactive with particular consistency. This well-established neural circuitry makes OCD an attractive candidate for novel anatomically targeted treatments. Indeed, invasive circuitry-based neurotherapeutics such as deep brain stimulation (DBS) and stereotactic ablation have been intensively investigated in OCD, and they show considerable promise for profoundly refractory cases.

Neurofeedback provides an innovative, noninvasive way to modulate the same circuitry. As a proof of concept, Applicant has developed an fMRI-based neurofeedback approach that is efficacious both in subclinically anxious individuals and, in pilot observations and an ongoing controlled study, in patients with OCD. Embodiments of the invention would make therapeutic neurofeedback more generalizable and accessible, which would be an important treatment advance.

### Neurofeedback

Neurofeedback is a specific form of biofeedback. In biofeedback, patients are given a real-time visual readout of physiological functions to which they would not normally have conscious access, such as heart rate or galvanic skin response (GSR), and they use this feedback to learn through trial and error to exert control over that physiological parameter. When the biofeedback signal is related to a pathological state, this can lead to increased control over that state and be of therapeutic benefit.

Real-time fMRI (rt-fMRI)-driven neurofeedback utilizes the same basic principles, but the biofeedback signal reflects the metabolic activity of a defined brain area. By giving subjects a visual readout of the activity of a specific brain region, neurofeedback enables them to learn via trial-and-error to control its activity. This can lead to altered functional connectivity within the targeted circuitry that persists even in the absence of ongoing efforts at control, as demonstrated herein.

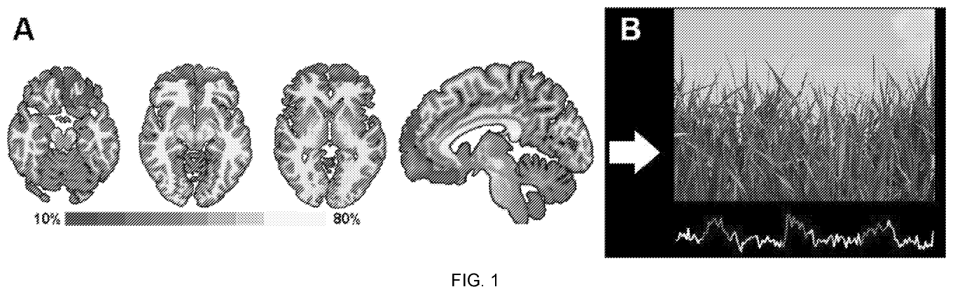

Applicant tested rt-fMRI neurofeedback targeting the OFC in subjects with high, but subclinical, contamination-related anxiety. Subjects first underwent an fMRI session during which they were shown neutral and contamination-anxiety-provoking images. Brain voxels in the OFC and frontal pole that were differentially responsive to contamination anxiety provocation were mapped in each subject. (Their distribution across subjects is shown in FIG. 1, Panel (A).) Subjects then spent a one-hour session with a trained psychotherapist, who gave them suggestions as to particular cognitive approaches that might be used to mitigate discomfort associated with such contamination-related images.

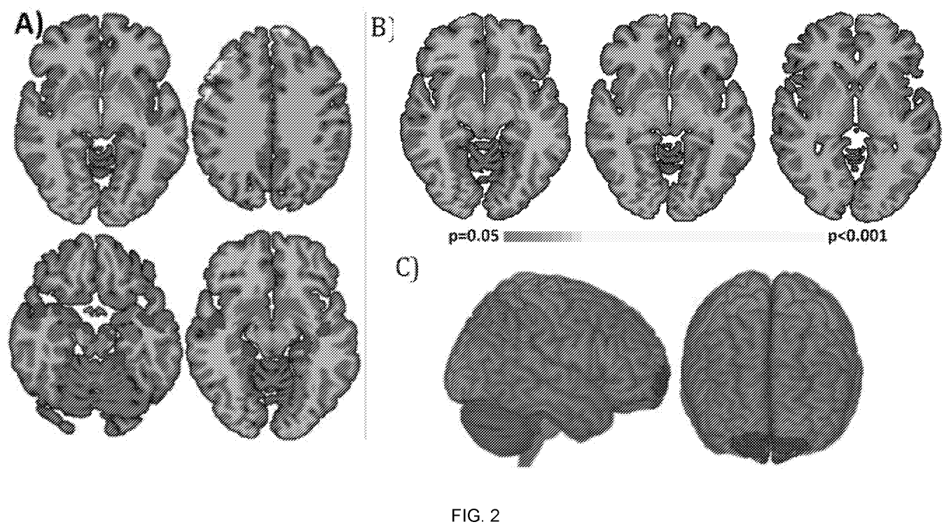

Following these preliminary steps, 10 subjects underwent two 90-minute sessions of neurofeedback. During neurofeedback, subjects were presented with either anxiety-provoking or neutral images, a readout of the current activity level in their OFC, and a color-coded arrow as depicted in FIG. 1, Panel (B). They were instructed to increase orbitofrontal activity when the arrow was red and pointed up, to decrease it when the arrow was blue and pointed down, and to relax when the arrow was white and pointed to the right. 10 additional subjects underwent sham neurofeedback: they saw an identical visual display, but the feedback line corresponded to brain activity from the matched neurofeedback subject, not their own, and therefore did not provide any true informative feedback. Over the course of 2 neurofeedback sessions, subjects receiving true neurofeedback improved their ability to control the OFC, while those receiving sham neurofeedback did not. Furthermore, those receiving true neurofeedback reported reduced image-driven anxiety several days after the procedure, while those who received sham neurofeedback did not. This difference was statistically significant. Neurofeedback success was predicted by global brain functional connectivity, a measure of brain network organization, in the OFC at baseline (FIG. 2, Panel (B)), and improvements in anxiety correlated with changes in this measure over the course of training (FIG. 2, Panel (C)).

Neurofeedback also produced striking changes in brain functional connectivity throughout the brain as depicted in FIG. 2, Panel (A). Several days after the completion of neurofeedback, individuals who received true neurofeedback showed reduced global brain functional connectivity in a broad network of limbic brain regions associated with anxiety, such as insula, ventral tegmental area, and amygdala. Resting-state connectivity was increased in the dorsolateral prefrontal cortex, which is associated with cognitive control. Importantly, these observations were made while subjects were at rest, not during anxiety provocation or effortful anxiety control. These changes, therefore, represent lasting alterations in brain organization, not the correlates of learned behaviors. Subjects who received sham neurofeedback showed no significant changes in brain resting-state connectivity.

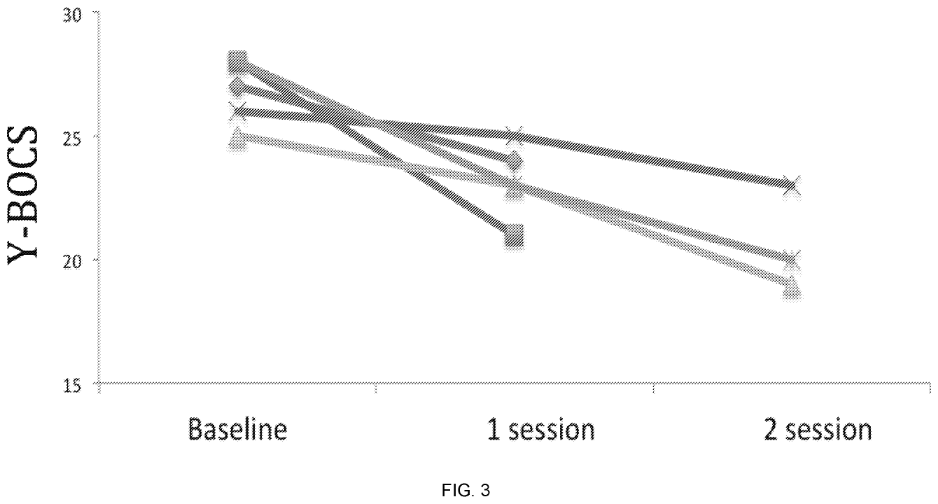

In an uncontrolled pilot study, five OCD patients have completed this fMRI neurofeedback protocol. These patients were selected for their prominent contamination obsessions and cleaning compulsions. The first two patients completed only a single session of neurofeedback, due to funding limitations, whereas the other three patients completed the full protocol (2 sessions of neurofeedback each) as depicted in FIG. 3. All five patients showed symptom improvement. Several volunteered that they liked the protocol, felt that it had helped them, and wished they could do it more.

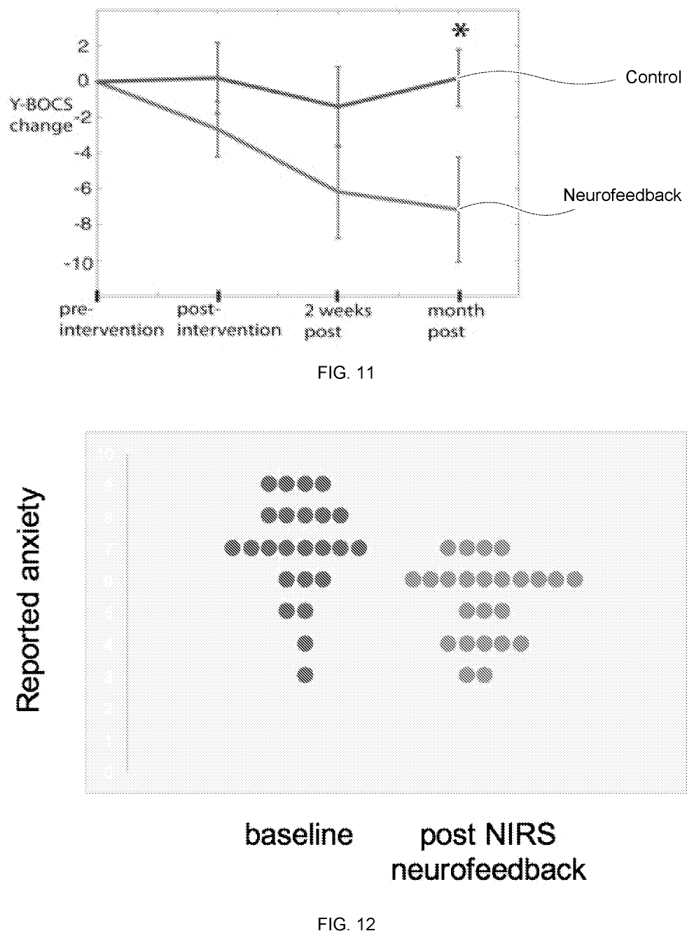

More recently, Applicant initiated a sham-controlled treatment study of individuals with OCD. Preliminary analysis of 12 subjects (6 real neurofeedback, negative control/sham neurofeedback) indicate significant clinical improvement from the intervention, which grows over time, as depicted in FIG. 11.

However, fMRI-based neurofeedback is unlikely to make a major clinical difference for a large number of patients, for purely practical reasons. It requires many hours in a multimillion dollar fMRI machine, a sophisticated computer system, and a dedicated staff, and is thus unlikely to become widely available.

fNIRS Neurofeedback

Aspects of the invention seek to address this drawback by adapting a much cheaper and more convenient technology to perform neurofeedback. Near-infrared spectroscopy (NIRS) measures changes in the concentration of oxy-hemoglobin (oxy-Hb) and deoxy-hemoglobin (deoxy-Hb), as well as changes in the redox state of cytochrome c oxidase, by measuring their different specific absorbance spectra in the near-infrared range using transcranial illumination. Functional NIRS (fNIRS) measurements, like fMRI, are based on the principle of neurovascular coupling: that is, brain activation leads to an increase in flow and, consequently, to an increase in the concentration of oxy-Hb and a decrease in the concentration of deoxy-Hb. Such changes are interpreted as a surrogate measure of local brain activity.

NIRS is especially suitable for psychiatric patients, for several reasons. First, it is relatively insensitive to motion artifact (in contrast to fMRI) and can be used in experiments in which motion might be expected. Second, subjects can be examined in a natural sitting position, in contrast to the physical constraints, discomfort, and artificial environment of fMRI machine. Third, the cost of this technique is much lower than that of other neuroimaging modalities, and implementation is straightforward. Fourth, the high temporal resolution of NIRS is useful in characterizing the time course of prefrontal activity in psychiatric disorders. Although NIRS has a lower spatial resolution than fMRI and cannot penetrate to deep brain structures, it can provide data concerning blood flow to the anterior region of the OFC. It has been used to assess brain function in a number of psychiatric disorders.

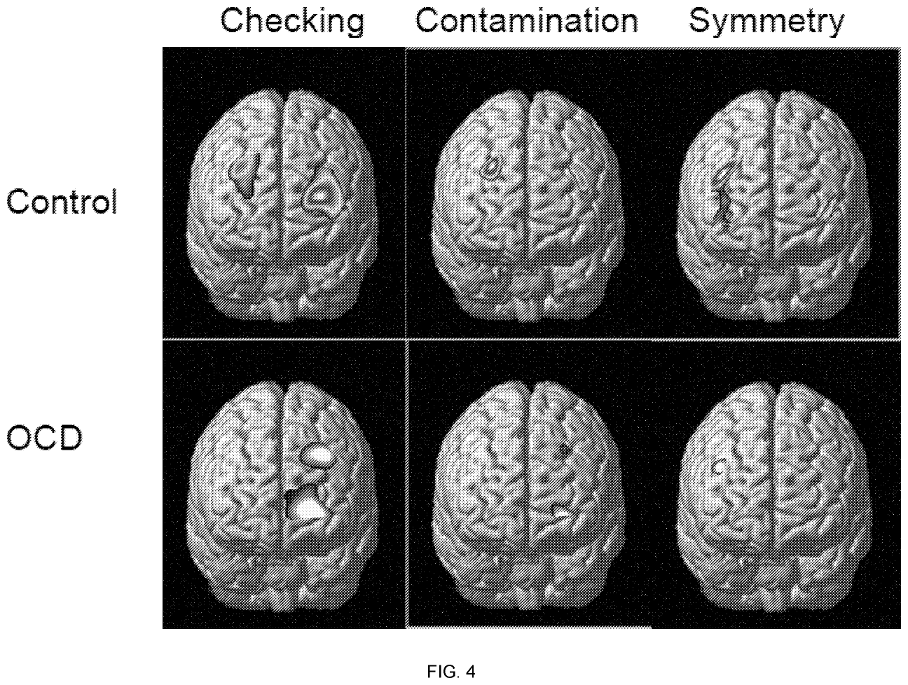

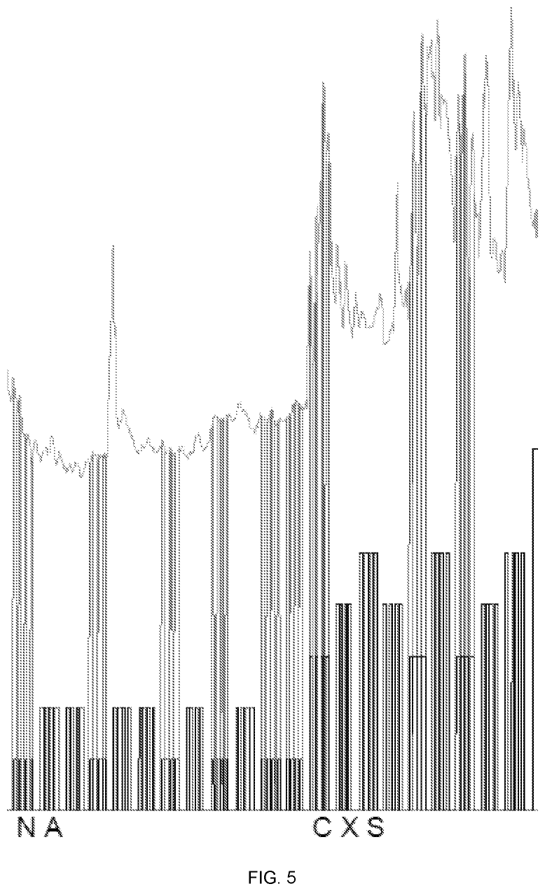

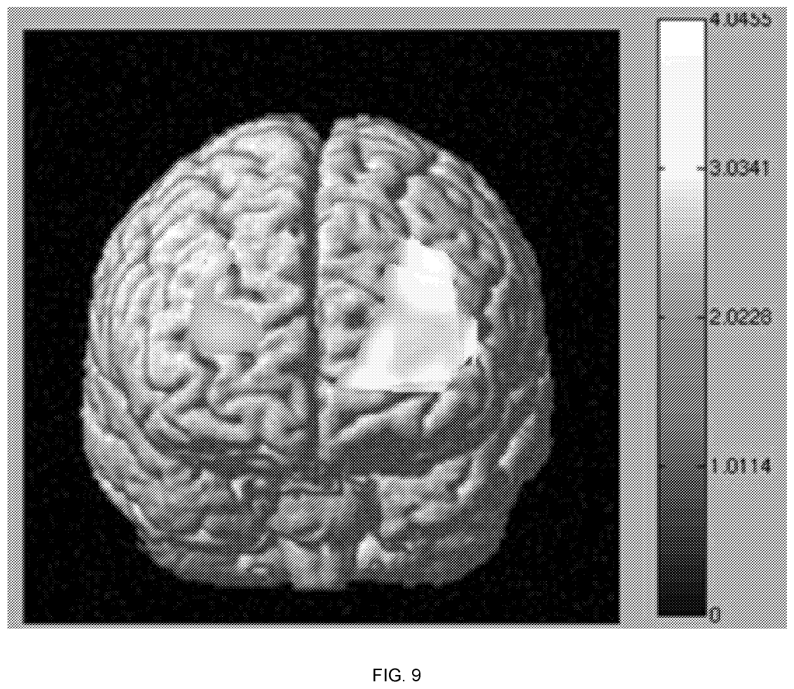

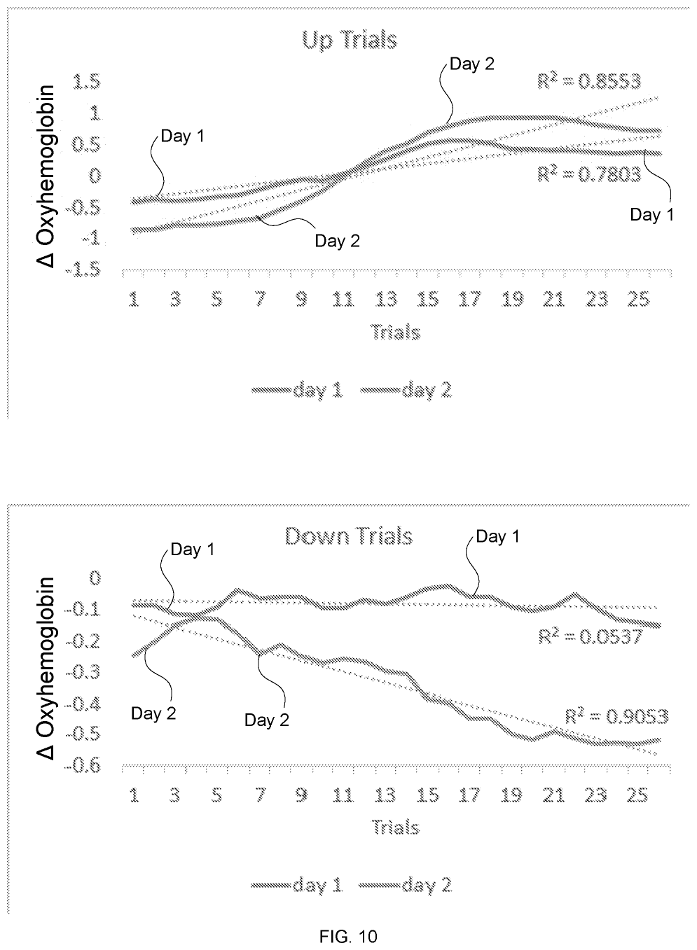

To demonstrate and optimize the ability of fNIRS to measure relevant frontal lobe blood flow changes in individuals with OCD, Applicant performed symptom provocation using visual stimuli in OCD patients and control subjects. Preliminary data depicted in FIG. 4 indicates a greater hemodynamic response (oxy-Hb) in the anterior lateral OFC/BA10 region in patients after symptom-related stimuli (compared to neutral images). These control subjects, who did not have measurable contamination anxiety, showed reduced frontal activation after symptom-related stimuli. These preliminary results demonstrate the fNIRS measurement of relevant hemodynamic changes in anterior regions of the OFC/frontal pole—the region used as a seed in the five OCD patients who underwent neurofeedback (depicted in FIG. 3)—in response to symptom-relevant stimuli. FIG. 5 presents raw fNIRS signal from the most symptom-related optode pair (i.e., an associated emitter optode and detector optode) in a single subject, showing the ability to distinguish symptom-related signal in real time. (This optode pair responds particularly well to contamination stimuli, shown in blue, relative to neutral stimuli, shown in red.) FIG. 9 presents grouped data and illustrates areas of the frontal lobe of the brain in which brain activity was greater in individuals with OCD than in controls, when confronted with symptom-provocative stimuli.

### Methods of Treating a Subject

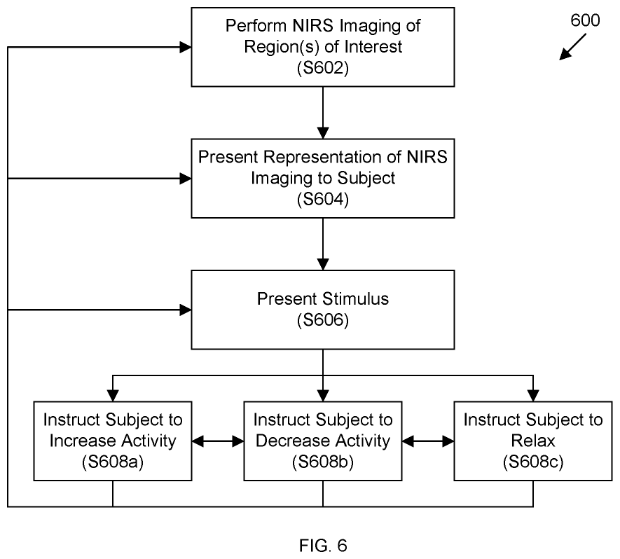

Referring now to FIG. 6, one embodiment of the invention provides a method 600 of treating a subject. This method 600 can be applied to a variety of mental disorders such as anxiety disorder, obsessive-compulsive disorder, and the like. Application to a particular subject can be based clinical diagnosis of a particular mental disorder, demonstrated symptomology (e.g., as measured using techniques such as the Yale-Brown Obsessive Compulsive Scale (Y-BOCS), the Hamilton Depression Rating Scale (HAM-D), the Beck Depression Inventory, the Hamilton Anxiety Rating Scale (HAM-A), and the like), and the like. Y-BOCS is described in publications such as W. K. Goodman et al., “The Yale-Brown Obsessive Compulsive Scale. I. Development, use, and reliability”, 46(11) Arch. Gen. Psychiatry 1006-11 (1989).

In step S602, near-infrared spectroscopy (NIRS) imaging of one or more regions of interest in the subject's brain is performed.

NIRS systems are commercially available from a variety of sources including Rogue Research Inc. of Montreal, Quebec; NIRx Medical Technologies, LLC of Los Angeles, Calif.; TechEn, Inc. of Milford, Mass.; Cortech Solutions, Inc. of Wilmington, N.C.; Shimadzu Corporation of Kyoto, Japan; and Hitachi Medical Systems America Inc. of Twinsburg, Ohio.

A NIRS cap can be positioned over the subject's frontal lobes using the international 10-20 system. Measurements of cortical perfusion can be obtained at 10 Hz using a fifty-two-channel near-infrared spectroscopy machine (ETG-4000, Hitachi Medical). To standardize the placement of the optode lattice, a source probe can be placed directly above the right ear in all participants. Following the acquisition of functional data, optodes can be removed from the lattice, and a 3D digitizer system can be used to localize the placement of each optode in relation to reference points on the subject's head (nasion, left and right ears, top and back of the head). The coordinate placements of each subject's channels and 5 reference points can be used to normalize the location of each recording channel into Montreal Neurological Institute (MNI) space for subsequent general linear model (GLM) group-level analyses.

In other embodiments, a 3D magnetic digitizer (available under the PATRIOT™ trademark from Polhemus of Colchester, Vt.) can be used to identify the optode position of each subject immediately before data collection to normalize the position of the individual channels of the NIRS cap to the shape of each subject's skull as discussed in M. Okamoto & I. Dan, “Automated cortical projection of head-surface locations for transcranial functional brain mapping,” 26 NeuroImage 18-28 (2005). Three-dimensional coordinates of anatomical landmarks on the head can be recorded in addition to locations of the individual optodes using procedures previously described in M. Okamoto et al., “Three-dimensional probabilistic anatomical cranio-cerebral correlation via the international 10-20 system oriented for transcranial functional brain mapping,” 21 NeuroImage 99-111 (2004). A digitizer pen can be used to indicate landmark positions of nasion, inion, T3, T4 and Cz according to the standard 10-20 coordinate system. After these anatomical landmarks are recorded, individual probe positions can be obtained. These coordinates can be used to estimate the position of each channel as defined by an emitter-detector optode pair and normalized to Montreal Neurological Institute (MNI) standard brain space coordinates using NIRS-SPM software. The MNI coordinates can be used to calculate probability of channel position using defined Brodmann's Areas and anatomical areas as indicated in the Talairach daemon.

Exemplary regions of interest for anxiety disorder and/or obsessive-compulsive disorder include the orbitofrontal cortex, and frontal pole. Other regions of interest can be selected for other mental disorders.

When a neurofeedback procedure in a particular embodiment is repeated on separate sessions or separate days, it is necessary to return the optodes to the same positions on successive days, such that the neurofeedback signal reflects the activity of the same brain regions on successive sessions. This may be done, for example, by placing optodes in a rigid array and orienting the array relative to anatomical landmarks by use of calipers, or, for example, by use of a 3D localization technology such as those described herein.

F-NIRS signaling can be collected at high frequency by some hardware, but the underlying neural signature is constrained by the hemodynamic response function, which describes the relationship between neural activity and measurable changes in blood flow. High frequency (greater than a few hertz) during neurofeedback thus can be downsampled to a frequency closer to the time scale of the hemodynamic response function (0.2-2 Hz) for more effective coupling with the presentation of neurofeedback stimuli.

In step S604, a representation of the NIRS imaging is presented to the subject. A variety of display devices can be utilized. For example, a display screen can be mounted in proximity to the subject. In another example, the representation is displayed by a head-mounted display such as those available under the OCULUS RIFT® trademark from OCULUS VR, LLC of Menlo Park, Calif. and the SAMSUNG GEAR VR® trademark from Samsung Electronics Co., Ltd. of Suwon-si, Republic of Korea. Such display devices can include a cathode ray tube (CRT), a plasma display, a liquid crystal display (LCD), an organic light-emitting diode display (OLED), a light-emitting diode (LED) display, an electroluminescent display (ELD), a surface-conduction electron-emitter display (SED), a field emission display (FED), a nano-emissive display (NED), an electrophoretic display, a bichromal ball display, an interferometric modulator display, a bistable nematic liquid crystal display, and the like.

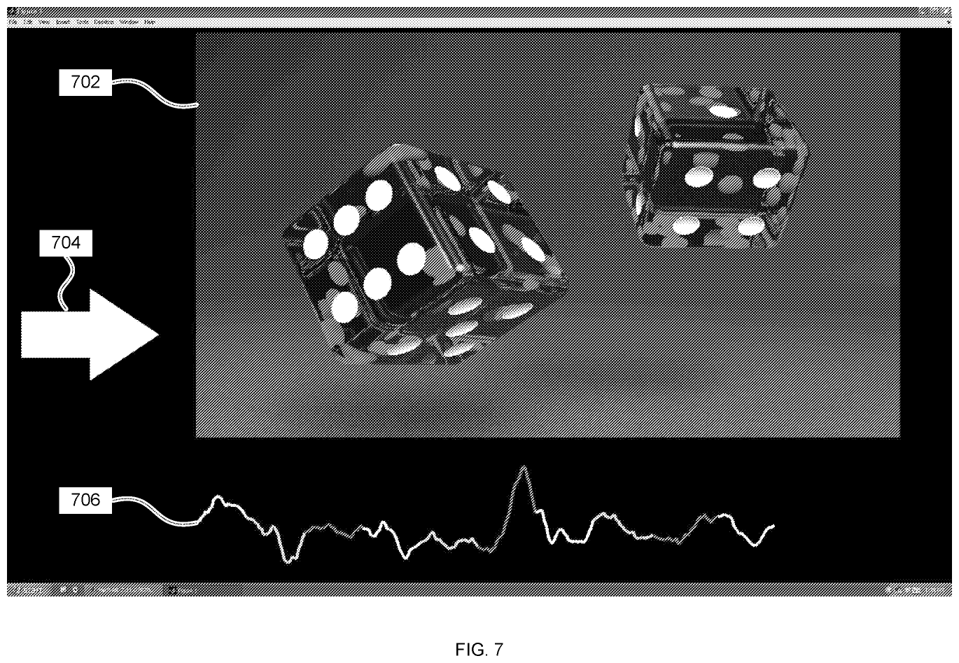

Exemplary graphic displays are depicted in FIG. 1, Panel (B) and FIG. 7. Such displays can include an image 702 (e.g., symptom-related, anxiety-inducing, and/or neutral images), instructions 704 regarding modulation of neural activity (e.g., an up arrow instructing the user to increase activity, a down arrow instructing the user to decrease activity, or a lateral arrow instructing the user to relax), and a representation of NIRS imaging 706. A variety of representations 706 can be utilized such as line graphs as depicted in FIG. 1, Panel (B) and FIG. 7. For example, the color of the line can change to reflect the instructions given during a particular epoch (e.g., by matching a color associated with a particular instruction and/or arrow direction). In other embodiments, a heat map is superimposed on one or more graphical representations of a brain. The displayed representation of NIRS imaging can present representation(s) of a single channel of data (e.g., relating to a particular location within the subject's brain, a particular optode, a particular wavelength, and the like) or a combination of multiple channels.

In step S606, one or more stimuli are presented. Such stimuli can be presented using the display devices described herein. Stimuli need not be exclusively graphical and can additionally or exclusively also include other forms such as auditory, tactile, olfactory, and the like. Stimuli can be symptom-related (e.g., anxiety-inducing) or neutral and can be pre-coded and/or selected based on a particular subject's mental disorder. For example, anxiety-inducing stimuli for a subject having contamination anxiety could include images of money, elevator controls, toilets, keys, telephones, and the like, while neutral stimuli can include images of animals, landscapes, children, and the like. Suitable images include those provided in the Maudsley Obsessive Compulsive Symptom Set described in D. Mataix-Cols et al., “The Maudsley Obsessive-Compulsive Stimuli Set: validation of a standardized paradigm for symptom-specific provocation in obsessive-compulsive disorder, 168(3) Psychiatry Res. 238-41 (2009) and the International Affective Picture System described in P. J. Lang et al., “International affective picture system (IAPS): Affective ratings of pictures and instruction manual,” Technical Report A-82008, University of Florida, Gainesville, Fla. (2008).

In step S608, the subject is instructed to modulate (e.g., by increasing or decreasing) activity in a region of interest or to relax. Instructions can be displayed graphically using the display device discussed herein. Additionally or alternatively, instructions can be audibly provided.

As seen in FIG. 6, one or more the method steps can occur simultaneously with others. For example, the stimulus can continue to be displayed (S606) after the subject is instructed to decrease activity (S608a) and while NIRS imaging continues to be performed (S602) and displayed (S604) as feedback that allows the user to further module their neural activity in the region of interest.

Likewise, the steps can be repeated multiple times in a single session. For example, the subject may be presented with a plurality of stimuli (e.g., 10, 20, and the like).

Additionally, the steps can be repeated across multiple sessions. For example, method can be performed weekly, biweekly, or at other intervals, whether fixed or variable.

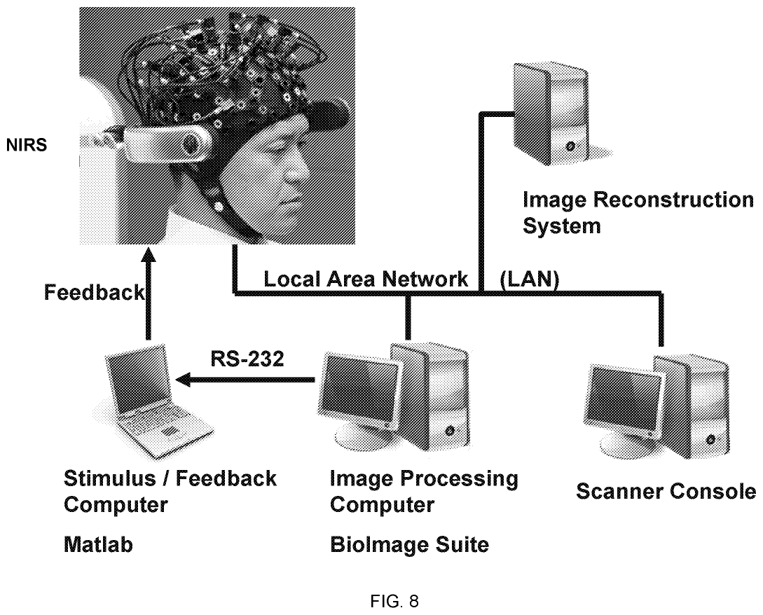

To illustrate this method, Applicant performed fNIRS neurofeedback on subjects with clinical or subclinical OCD symptoms. An example of the presentation of feedback data to a subject undergoing fMRI neurofeedback is provided in FIG. 7. The system used in this instantiation of the invention is illustrated in FIG. 8. fNIRS data showing a single OCD subject's ability to learn to control OFC activity over the course of two sessions of fNIRS neurofeedback is shown in FIG. 9; ability to control activity during both ‘up’ blocks (top panel) and ‘down’ blocks (bottom panel) was improved in the second neurofeedback session compared to the first session, indicating learning. Data from an individual with subclinical OCD illustrating that fNIRS neurofeedback is of benefit to anxiety symptoms is shown in FIG. 12; anxiety reported upon presentation of symptom-provoking images before neurofeedback (left, blue) was decreased after neurofeedback (right, orange).

Implementation in Computer-Readable Media and/or Hardware

The methods described herein can be readily implemented in software that can be stored in computer-readable media for execution by a computer processor. For example, the computer-readable media can be volatile memory (e.g., random access memory and the like) and/or non-volatile memory (e.g., read-only memory, hard disks, floppy disks, magnetic tape, optical discs, paper tape, and the like).

Additionally or alternatively, the methods described herein can be implemented in computer hardware such as an application-specific integrated circuit (ASIC).

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of treating a subject diagnosed with one or more mental disorders selected from the group consisting of: anxiety disorders, mood disorders, trauma-associated disorders, psychotic disorders, and obsessive-compulsive disorder and related disorders, the method comprising:
performing near-infrared spectroscopy (NIRS) imaging of one or more regions of interest in the subject's brain;
presenting a representation of the NIRS imaging to the subject; and
while continuing to perform the performing and presenting steps:
presenting a stimulus of anxiety or other symptomatology to the subject;
instructing the subject to increase activity in the one or more regions of interest; and
instructing the subject to decrease activity in the one or more regions of interest.

2. The method of claim 1, wherein the one or more regions interest include one or more selected from the group consisting of: an orbitofrontal cortex, a frontal pole, and a basal ganglia.

3. The method of claim 1, wherein the one or more regions interest include the subject's frontal pole.

4. The method of claim 1, wherein the representation of the NIRS imaging is a graphical representation.

5. The method of claim 4, wherein the graphical representation is a chart.

6. The method of claim 1, further comprising:
downsampling data from the NIRS imaging to between about 0.2 Hz and about 6 Hz.

7. The method of claim 1, further comprising:
repeating all steps within a single session.

8. The method of claim 1, further comprising:
repeating all steps in a new session.

9. The method of claim 8, further comprising:
calibrating the NIRS imaging to image the one or more regions of interest in the subject's brain images in a previous session to ensure that similar brain regions are monitored in successive sessions.

10. The method of claim 1, wherein obsessive-compulsive disorder and related disorders comprises one or more selected from the group consisting of: obsessive-compulsive disorder, body dysmorphic disorder, hoarding disorder, trichotillomania, excoriation, and skin-picking disorder.
